(19) United States
(12) Patent Application Publication
(10) Pub. No.: US 2018/0139366 A1
(43) Pub. Date: May 17, 2018

(54) SYSTEM AND METHOD FOR LIGHT SHEET MICROSCOPE AND CLEARING FOR TRACING

(71) Applicant: Cold Spring Harbor Laboratory, Cold Spring Harbor, NY (US)

(72) Inventors: Arun Narasimhan, Huntington Station, NY (US); Judith Mizrachi, Flushing, NY (US); Kannan Umadevi Venkataraju, Floral Park, NY (US); Dinu F. Albeanu, Huntington, NY (US); Pavel Osten, Brooklyn, NY (US)

(21) Appl. No.: 15/811,234

(22) Filed: Nov. 13, 2017

Related U.S. Application Data
(60) Provisional application No. 62/421,012, filed on Nov. 11, 2016.

(51) Int. Cl. H04N 5/225; A61B 1/00

(52) U.S. Cl. CPC H04N 5/2256; H04N 2005/2255; A61B 1/00193

(57) ABSTRACT

An exemplary system and method for imaging tissue includes using an illumination objective, directing one or multi photon excitation lights onto a portion of a tissue from a position on top and at an oblique angle relative to the tissue while the tissue is mounted on a stage. The method further includes generating a tissue-penetrating light-sheet from the one or multi photon excitation lights. Using a detection objective, the method detects the tissue-penetrating light-sheet. Upon detecting the tissue-penetrating light-sheet, it uses the detection objective, to collect fluorescent signals from the tissue and uses the fluorescent lights to acquire a first image of the tissue while the tissue is an imaging position. A second image of the tissue is acquired while the tissue is in the imaging position. The first and second images each defined by first and second data, respectively. Subsequently, the tissue is moved to a sectioning position and with the use of an integrated Vibratome, a portion of the tissue, with known thickness, is sectioned. The process repeats until the tissue, in its entirety, has been sectioned, with images acquired each time. Image data, from the acquired images, are stitched to create a 3-dimensional 3D) image of the tissue.

### CROSS REFERENCE TO RELATED APPLICATIONS

This application claims priority to U.S. Provisional Application No. 62/421,012, filed on Nov. 11, 2016, by Arun Narasimhan, et al., and “System And Method For Light Sheet Microscope And Clearing For Tracing”.

### STATEMENT OF FEDERALLY FUNDED SPONSORSHIP

This invention was made with government support under U01 MH105971 and R01 MH096946 awarded by the National Institutes of Health. The government has certain rights in the invention.

### BACKGROUND

Some companies integrate 2-photon microscopy and tissue sectioning in a method called serial two-photon tomography (STPT). However, this method can be slow and the cost of the instrument can be high. “Traditional” light-sheet fluorescence Microscopes (LSFM) either image at high resolution but small volume tissues or image large volume tissue but at low resolution.

### SUMMARY

A system and method for imaging tissue can include using an illumination objective, directing one or multi photon excitation lights onto a portion of a tissue from a position on top and at an oblique angle relative to the tissue while the tissue is mounted on a stage. The method further includes a tissue-penetrating light-sheet, from the one or multi photon excitation lights. Using a detection objective, the method detects the tissue-penetrating light-sheet. Upon detecting the tissue-penetrating light-sheet, using the detection objective, the fluorescent signals from the tissue are collected and used to acquire a first image of the tissue while the tissue is an imaging position. Further, a second image is acquired of the tissue while the tissue is in the imaging position. The first and second images each defined by a first image and second data, respectively. Subsequently, the tissue is moved to a sectioning position where using an integrated Vibratome, the portion of the tissue is sectioned and the first and second images are stitched to create a 3-dimensional 3-D) image of the tissue. The acquiring steps through the stitching step are repeated until all portions of the tissue have been sectioned and imaged.

This Summary is provided merely for purposes of summarizing some example embodiments so as to provide a basic understanding of some aspects of the disclosure. Accordingly, it will be appreciated that the above described example embodiments are merely examples and should not be construed to narrow the scope or spirit of the disclosure in any way. Other embodiments, aspects, and advantages of various disclosed embodiments will become apparent from the following detailed description taken in conjunction with the accompanying drawings which illustrate, by way of example, the principles of the described embodiments.

### DRAWINGS

FIG. 1 shows a flow chart 100 of steps generally performed to image a tissue.

FIGS. 2a-2f show the steps generally performed during slicing by the imaging system of various embodiments.

FIG. 3 shows a picture of an imaging system in accordance with an exemplary implementation of the invention.

FIG. 4 shows a close-up picture of the parts of each of two objectives housing a microscope.

FIG. 5 shows a close-up picture of an imaging system in an exemplary embodiment of the invention.

FIG. 6 shows the imaging system of FIG. 5 with laser effects during the operation of the imaging system.

FIG. 7 shows, in conceptual form, various stages for generating image data by an imaging system of exemplary implementations of the invention.

FIG. 8 shows a microscope imaging system 80, in accordance with an exemplary implementation of the invention.

FIG. 8a shows the microscope imaging system of FIG. 8 coupled to a processing circuit.

FIG. 9 shows a conceptual view of the Vibratome 34 mounted to the post 32 of FIG. 3.

FIG. 10 shows a flow chart of some of the steps performed when mounting the tissue.

FIG. 11 shows the tissue mounted, in accordance with an exemplary implementation of the invention.

FIG. 12 shows a flow chart of some of the steps generally performed in determining volumetric imaging parameters at the outset of the imaging operation.

FIG. 13 shows a block diagram of some of the structures of the imaging system of an exemplary implementation.

FIG. 14 shows, in conceptual form, a part of the imaging system of an exemplary implement of the invention.

FIGS. 15 and 16 show, in conceptual form and flow chart form, respectively, the process of moving the excitation light across the tissue during volumetric measurement.

FIG. 17 shows various magnifications of 3D images of a mouse brain tissue generated by an imaging system of an implementations of the invention.

FIG. 18 shows a block diagram of the processor circuit of an implementation of the invention.

### DETAILED DESCRIPTION

A method of imaging tissue includes using an illumination objective, directing one or multi photon excitation lights onto a portion of a tissue from a position on top and at an oblique angle relative to the tissue while the tissue is mounted on a stage. The method further includes generating a tissue-penetrating light-sheet from the one or multi photon excitation lights. Using a detection objective, the method detects the tissue-penetrating light-sheet. Upon detecting the tissue-penetrating light-sheet, it uses the detection objective, to collect fluorescent signals from the tissue and uses the fluorescent lights to acquire a first image of the tissue while the tissue is an imaging position. A second image of the tissue is acquired while the tissue is in the imaging position. The first and second images each defined by first and second data, respectively. Subsequently, the tissue is moved to a sectioning position and with the use of an integrated Vibratome, a portion of the tissue, with known thickness, is sectioned. The process repeats until the tissue, in its entirety, has been sectioned, with images acquired each time. Image data, from the acquired images, are stitched to create a 3-dimensional (3D) image of the tissue.

Alternatively, the method includes chemical clearing of the tissue prior to starting sectioning. Optionally, the method includes moving the stage, by electronic control, locating the center of a top surface of the tissue, prior to sectioning and imaging. In yet another exemplary method, images are acquired with image sensors that may be charge coupled device or CMOS imaging device. Optionally, the method includes, in addition to electronic control of the stage, controlling movement of the Vibratome using a processor.

Further and optionally, a type of light sheet fluorescence microscope (LSFM) is described in which a thin laser light is directed from an objective to produce a light sheet in a plane that is orthogonal to the detection plane and at an oblique (for example about 45-degree) angle above the tissue. The tissue is imaged after using another objective, also positioned over the top of the tissue, orthogonal to the detection illumination plane and at an oblique (for example about 45-degree) angle above the tissue, receives fluorescent images from the tissue upon the generation of the light sheet. Once the tissue is imaged from the top, the imaged tissue is mechanically removed (for example by sectioning with a tissue Vibratome), and the process is repeated until the entire tissue is completely imaged. Thus, this new type of a light sheet fluorescence microscope integrates fast tissue imaging by light sheet fluorescence microscopy and mechanical sectioning that keeps the optical conditions (also referred to herein as “optical parameters” or “optical imaging parameters”) constant throughout the whole tissue and allows the use of high magnification/high NA objectives. In some example, given the oblique illumination plane, the instrument can be referred to as oblique light-sheet tomography (OLST) or oblique light-sheet microscopy (OLSM). Additionally or alternatively, related software can provide for super-resolution of the imaged data by applying super-resolution optical fluctuation imaging (SOFI) to the light-sheet fluorescence data, which utilizes optical fluctuation for cumulant analysis to achieve super-resolution. While SOFI has been applied with other imaging modalities, for example confocal microscopy, this is the first application of SOFI with single-photon light-sheet microscopy. The SOFI application can be referred to as oblique light-sheet tomography or microscopy at super-resolution (OLSTsr or OLSMsr).

Imaging whole tissues in 3D can be used in various scientific and medical fields. For example, in neuroscience 3D imaging is used to better understand brain anatomy and connectivity in animal models or in 3D cell cultures called organoids. Another application is in medicine for inspections of cancer tissue, either human cancer tissue taken for diagnosis or xenografts of cancer tissue in animal models. Presently, there are several commercial instruments for 3D tissue-imaging. The OLST and OLSTsr are the only instruments that can image large tissue at both high light-resolution and super-resolution.

The first and/or second objective can each be Oblique Light Sheet Tomography (OLST); Light Sheet Fluorescence Microscope (LSFM); Principle of LSFM; Advantages; Clearing (0MCS); Example protocol; Other Examples; and example Cleared Thy1GFP mouse brain.

A system, method and/or microscope for imaging tissue can includes using an illumination objective, directing one or multi photon excitation lights onto a portion of a tissue from a position on top and at an oblique angle relative to the tissue while the tissue is mounted on a stage. The method further includes generating a tissue-penetrating light-sheet from the one or multi photon excitation lights. Using a detection objective, the method detects the tissue-penetrating light-sheet. Upon detecting the tissue-penetrating light-sheet, it uses the detection objective, to collect fluorescent signals from the tissue and uses the fluorescent lights (signals) to acquire a first image of the tissue while the tissue is an imaging position. A second image of the tissue is acquired while the tissue is in the imaging position. The first and second images each defined by first and second data, respectively. Subsequently, the tissue is moved to a sectioning position and with the use of an integrated Vibratome, a portion of the tissue, with known thickness, is sectioned. The process repeats until the tissue, in its entirety, has been sectioned, with images acquired each time. Image data, from the acquired images, are stitched to create a 3-dimensional 3D) image of the tissue.

In an exemplary implementation, the bath chamber includes “chemical clearing” to aid in making the tissue transparent that results in acquiring a better quality 3-D image, particularly for tissues with large thicknesses.

In some aspects, the microscope includes a single-photon light-sheet microscope. In some aspects, the excitation light has a penetration depth in the tissue in the range of hundred micrometers or more because of a “chemical clearing” of the tissue by matching the refractive index of the tissue and the imaging solution. In some aspects, the excitation light has a penetration depth in the tissue in the range of hundred micrometers or more because of the use of multi-photon microscopy excitation. In some aspects, a fluorescent image is further detected. In some aspects, the microscope includes a multi-photon light-sheet microscope. In some aspects, the microscope includes Bessel beam light sheet microscope, or Airy beam light sheet microscope. In some aspects, wherein the microscope includes Swept, Confocally-Aligned Planar Excitation (SCAPE) microscope. In some aspects, the microscope employs a cylindrical lens and 3D astigmatic PSF deconvolution to improve the z-resolution. In some aspects, a fluorescent image is detected by light field camera, for example by one that uses an array of micro-lenses placed in front of an otherwise conventional image sensor to sense intensity or by multi-camera array. In some aspects, the sectioning further includes a Vibratome or other mechanical system that is integral with the microscope. In some aspects, the sectioning includes moving the stage from an imaging position to a sectioning position, removing a layer of tissue with a sectioning tool, and moving the stage to the imaging position. In some aspects, the moving comprises translating the stage in an X-Y plane and elevating the stage to position the tissue relative to the sectioning tool. In some aspects, further performing a plurality of sectioning to remove successive layers of tissue. In some aspects, further including programming a computer (or processor) to control an imaging sequence and a stage translation sequence. In some aspects, further detecting images with an image sensor. In some aspects, further detecting images with a charge coupled device or CMOS imaging device. In some aspects, the acquired images are further processed by Super-Resolution Optical Fluctuation Imaging (SOFI) analysis in order to enhance the resolution of the obtained images.

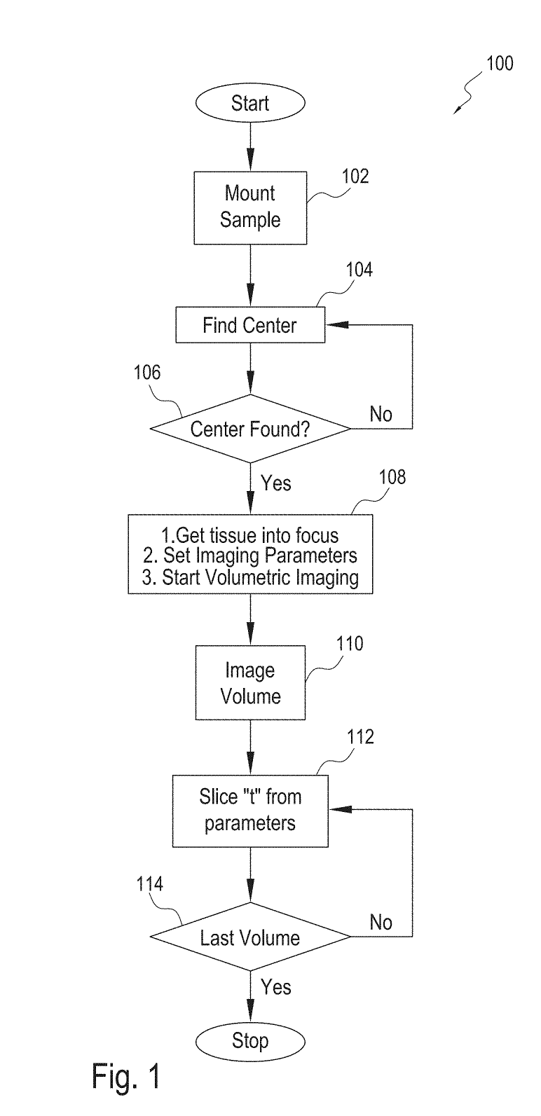

FIG. 1 shows a flow chart 100 of steps generally performed to image a tissue. It is understood that while certain steps are shown in FIG. 1 and/or discussed herein, other steps may be performed or one or more steps may be absent in various exemplary embodiments of the invention. At step 102, the tissue is mounted onto an agarose block, inside a bath chamber. The agarose block, with the tissue, is positioned on a metal plate and the metal plate is attached to motorized and computer-controlled stages, such as X, Y, Z.

Next at step 104, an attempt is made at locating the center of the (top) surface, facing the Vibratome, relative to the illuminating and detecting objectives. If the center is not located, such as determined at 106 in FIG. 1, the process continues to move the stage, onto which the tissue is mounted, as many times as it takes to locate the center of the surface. Once the center is found, the process continues to step 108.

At step 108, the tissue is manually brought into focus using, in an exemplary embodiment, imaging parameters (also referred to herein as “volumetric imaging parameters”, which are saved in a processor circuit. While the tissue is at an imaging position, volume imaging is performed at step 110, in FIG. 1, where the tissue, in its current state without the portion sliced at step 104, is imaged, per exemplary implementations of the invention. An exemplary volume imaging, such as the method of, without limitation, manual measuring may be employed. The tissue is moved from an imaging position to a slicing position, in close proximity to a Vibratome, if not already at the sectioning position.

Next, at step 112, while at an imaging position, the imaged tissue, of step 110, is sliced at a thickness, represented by “t”. The thickness of the sliced portion of the tissue may be among one of the imaging parameters.

Next, a determination is made by the processor as to whether or not the last (volume) portion of the tissue has been sliced and if so, the process stops, otherwise, the process repeats step 112 where slicing of a subsequent volume is performed until all volumes are determined to have been sliced by the processor, at 114. That is, successive slicing may be performed to ensure penetration deep into the layers of the tissue. In an exemplary embodiment, the tissue thickness is a function of the type of tissue being imaged. This allows for the creation of reliable image data even for tissues with large thicknesses.

At step 112, the tissue is physically sliced (during sectioning) at the predetermined thickness represented by “t” where a portion of the tissue is cut by a Vibratome, after the tissue has been moved in an in-plane direction (right or left), toward the Vibratome, and out-of-plane direction (elevated or lowered) relative to the Vibratome—sectioning position.

It is noted that during slicing, an image data is generated of the tissue in its current state, with a cutout. Ultimately, the image data of all slicing step are combined to form a 3D image of the tissue. The number of times slicing is performed is generally a function of the type of tissue employed. For example, a tissue taken from the liver is of a different type and may require a different number of slicing steps as opposed to tissue taken from the kidney.

An exemplary tissue size, one that comes from a mouse's brain can be approximately 1.5 centimeters (cm).

In a scenario where the image of the tissue spans beyond the surface of the tissue, during imaging, the imaging system is operating inefficiently and in a scenario where the image of the tissue is smaller than the surface of the tissue, the imaging system will likely be missing imaging of some portion of the tissue. It is therefore desirable for the image to be as close to the size of the tissue as possible.

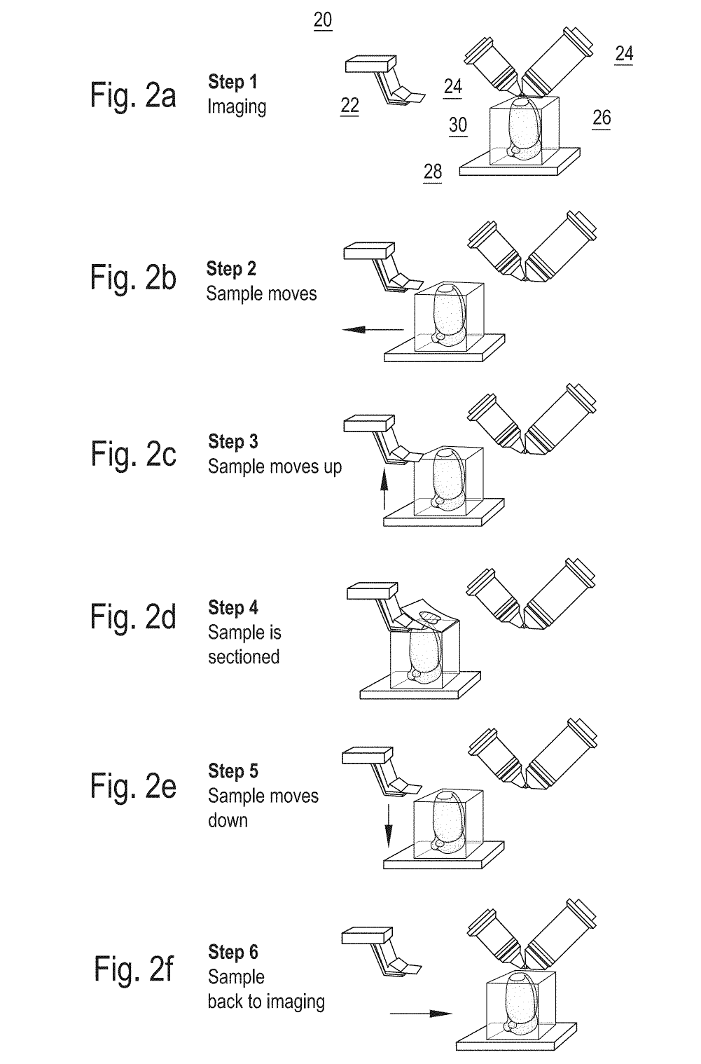

FIGS. 2a-2f show the steps generally performed during slicing by the imaging system of various embodiments. In each step, a Vibratome 22 is shown located in close vicinity to the imaging system 20. The imaging system 20 is shown to include two objectives, located on top and at an oblique angle relative to the tissue, as noted above. In addition to the tissue 30, a metal plate 28, and a block 26 are shown included in the imaging system 20. In an exemplary implementation, the block 26 is an Agarose block although other suitable blocks are contemplated. Block 26 is effectively used as a substrate onto which the tissue is formed or placed.

One of the objectives 24 generally serves as an illumination objective while the other serves as a detection objective, as will be explored in greater detail below.

In FIGS. 2a through 2f, the tissue 30 is shown to be embedded in block 26 and the block 26 is shown position on top of the metal plate 28, which is glued onto an X, Y, Z stage and moves when the stage moved under the direction of a processor.

At step 1, in FIG. 2a, imaging is performed prior to slicing where image data of the tissue, in its current state, is acquired. Next, at step 2, in FIG. 2b, the tissue 30, block 26 and metal plate 28 are moved to the left, closer to the Vibratome 22, as the stage moves to the left. This is in preparation for slicing where a portion from the top surface of the tissue is removed from by the Vibratome 22. An example of a commercially-available Vibrotome suitable for utilization in the imaging system 20 is VT1105 made by Leica Biosystems, Inc. of Illinois, US.

Integration of a Vibratome into an imaging system, for example of implementations of implementations of the invention allows for a better impinging quality. With an integrated Vibratome, light from a microscope with, for example 10× magnification, that otherwise would not travel deeper into the tissue with a large volume, can actually penetrate the entire tissue therefore allowing for quality imaging.

Once the light, in optical path 106, penetrates the tissue, it is scattered and a detection objective is used to collect fluorescent images generated therefrom.

The two distinct optical path 106 and 110 are generally aligned at a particular point and therefore quite bright when focused at that point with little to no focus away from the point. When both points, each from one of the objectives, are focused on the tissue, at generally the same point, they are considered aligned. Adjustment of the points may be made mechanically or otherwise. Once in focus, there is no longer a need to change the points and the objectives can be locked in, for example by physically screwing them into place. To achieve uniform optical parameters while maintaining high quality imaging, two objectives with the same or different magnifications may be employed. Examples of optical parameters include the power of the laser and the thickness of the light sheet.

In operation, laser (a combination of at least two lasers with distinct wavelengths) travels through optical pieces and thereafter undergoes 10× magnification by the illumination objective and fluorescent signals are ultimately collected by the 16× detection objective. The tissue emits different color lights, in the form of fluorescent signals, when arriving through the illumination optical path.

In an exemplary implementation, image and cut out sizes are nearly optimally set, or known as optimal conditions, because the size of the imaged tissue is known and the size of the desired size of the slice being cut is also known. The number of cutouts (or “slices”) is generally based on the type of tissue, i.e., lung vs. liver.

Next at step 3, in FIG. 2c, tissue 30, along with block 26 and metal plate 28, is elevated so as to be physically closer to the Vibratome 22 in preparation for sectioning (sectioning position). Subsequently, at step 4, in FIG. 2d, tissue 30 is sectioned using the Vibratome 22 at the sectioning position. Upon completion of sectioning, at step 5, in FIG. 2e, tissue 30 is lowered relative to the Vibratome 22 and at step 6, in FIG. 2f, it is moved to the right relative to the objectives 24, back to the imaging position. Movement of the tissue 30, block 26, and metal plate 28 is typically motor-driven, and electronically controlled, for example under the direction of a processor. Alternatively, such movement can be performed manually or through other techniques.

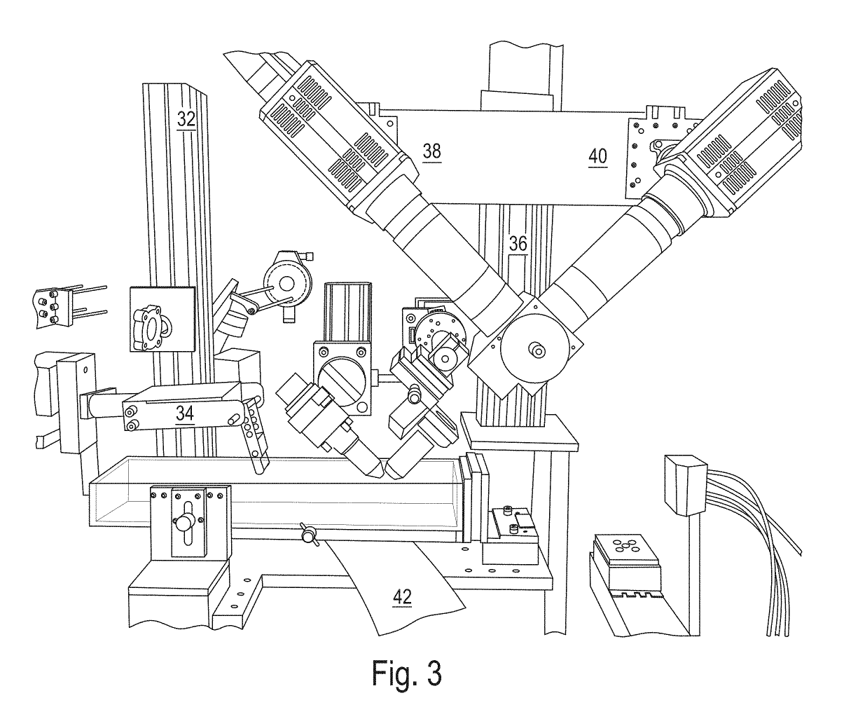

FIG. 3 shows a picture of an exemplary implementation of an imaging system, in accordance with implementation of the invention. In FIG. 3, the Vibratome is shown mounted to the post 32 at a location that is above the bath chamber 42. The wires shown to the left of the Vibratome, looking into the page, couple the Vibratome to a processor (not shown in FIG. 3) for electronic control. Two objectives 38 and 40 are shown mounted to post 36 with the post 36 extending to either side of the objectives 38 and 40 although this or other positioning discussed herein are merely exemplary implementations and not limiting. The objective 38 is generally used for illumination while the objective 40 is generally used for detection.

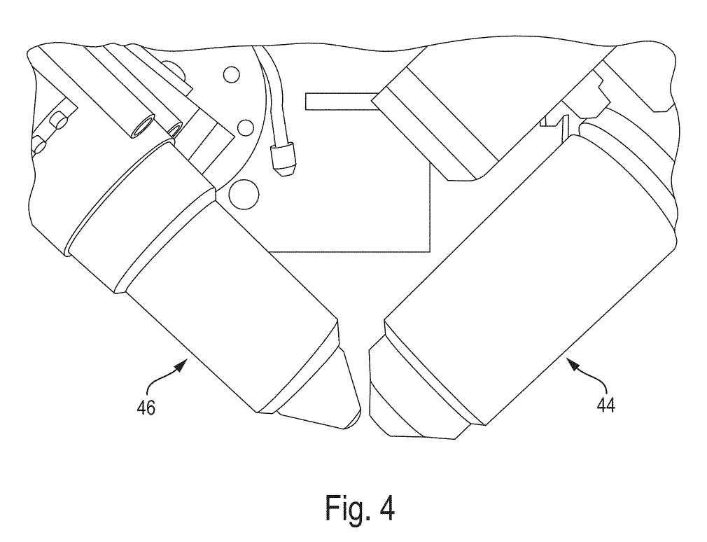

FIG. 4 shows a close-up picture of the parts of each of two objectives housing a microscope. The illumination and detection objectives 44 and 46, respectively, are shown positioned at an angle generally less than ninety degrees relative to one another. The objectives are locked in a focused position using metal posts, plates, and holders, as shown in FIG. 4.

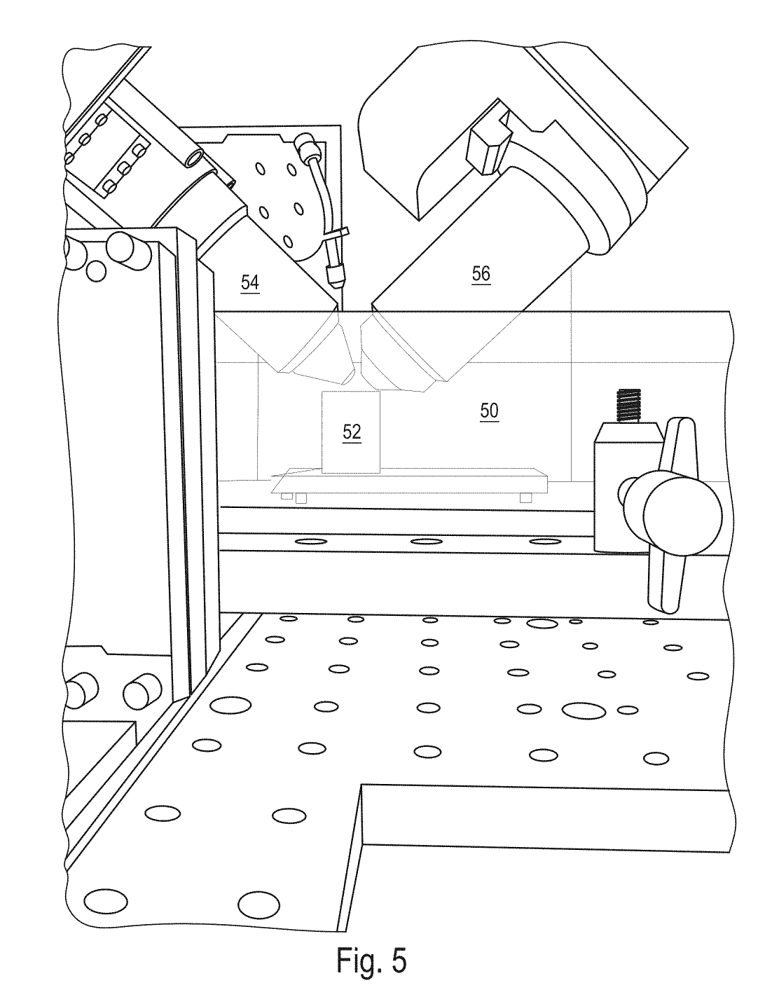

FIG. 5 shows a close-up picture of an imaging system in an exemplary embodiment of the invention. The tissue 52 is shown housed in the bath chamber 50 and mounted to a stage where the objectives 54 and 56 are shown imaging the tissue 52. As will be further explored, the objective 54 typically generates a laser beam that is ultimately scattered and filtered generating a line sheet to the tissue 52. The objective 56 typically collects fluorescent images from various stages of sectioning of the tissue 52.

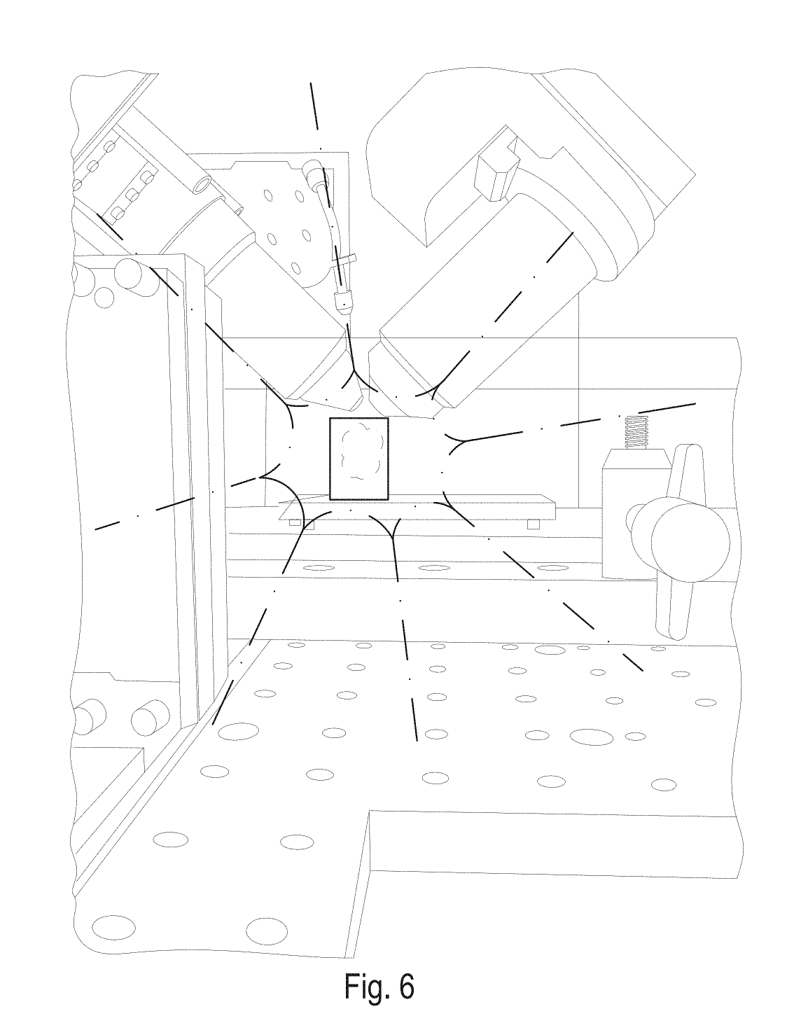

FIG. 6 shows the imaging system of FIG. 5 with laser effects during the operation of the imaging system.

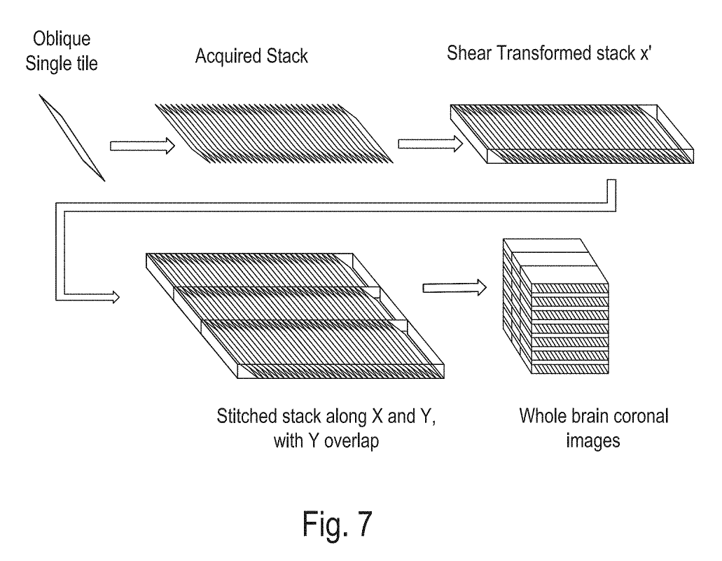

FIG. 7 shows, in conceptual form, various stages for generating the image data, performed by the imaging system of exemplary implementations of the invention. The steps of FIG. 7 are generally performed by a processor. Starting from the top left, an oblique single tile is generated and a stack of such tiles is acquired as a stack. A single stack is therefore acquired through the step of “stitched stack along X’ in FIG. 7, a part of the image data. These steps are repeated to create a second image data and all remaining images. Once all image data is collected, they are stitched, such as the “stitched stack along X and Y, with overlap” acquiring an image of a “whole brain coronal”, for instance. Exemplary commercially-available products suite for performing stitching is “imageJ” or “TeraStitcher” by the National Institute of Health.

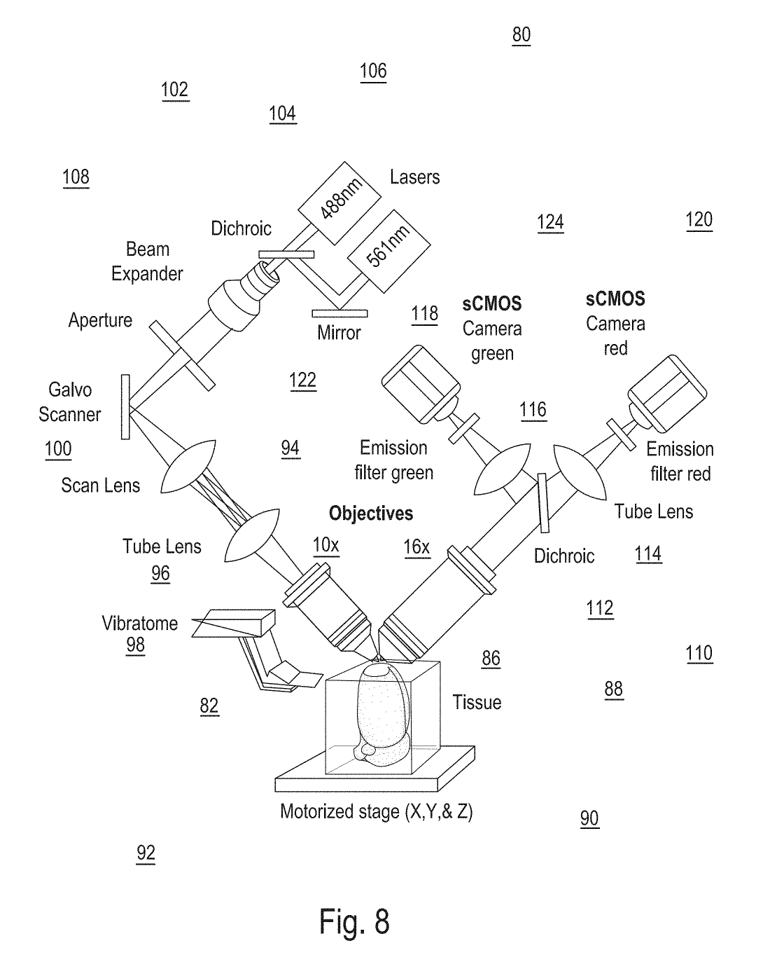

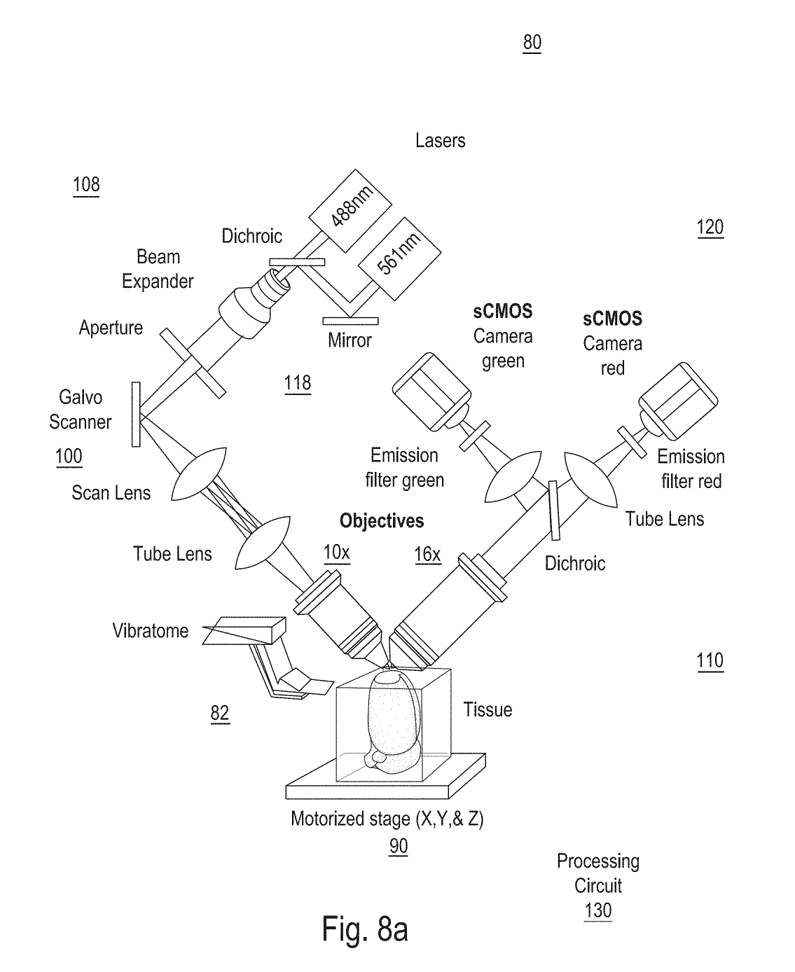

In FIG. 7, the “Oblique Single Tile” is the image acquired by the imaging system of various implementations of the invention. One image is generated per an imaging sensor (such as a camera for green color) and another is generated by another imaging sensor (such as a camera for red color), examples of which are shown in FIGS. 8 and 8a.

“Chemical clearing” is a process by which the tissue is made more transparent. An “imaging solution” is typically employed to do so. The tissue is bathed with chemical clearing, such as in a bath chamber. Examples of such a solutions are provided in U.S. Provisional Application No. 62/421,012, filed on Nov. 11, 2016, by Arun Narasimhan, et al., and entitled “SYSTEM AND METHOD FOR LIGHT SHEET MICROSCOPE AND CLEARING FOR TRACING”, the disclosure of which is incorporated herein as though set forth in full.

FIG. 8 shows a microscope imaging system 80, in accordance with an exemplary implementation of the invention. It is noted that the optical pieces shown in FIG. 8 may be replaced by other pieces suitable for the operation of imaging system 80. In FIG. 8, the microscope imaging system (also referred herein as “imaging system”) 80 is shown to include objectives 108 and 110 positioned over and on top of the tissue 86, at an oblique angle (for example approximately 45 degree) with the (illumination) objective 108 functioning as a tissue-penetrating light-sheet.

Tissue 86 is shown embedded in block 88, which is mounted to the metal plate 90 and the metal plate 90 is glued or in some other manner attached to the stage 90. Tissue 86 is currently shown to be in an imaging position. The Vibratome 82 is positioned in close proximity to the tissue 86 to allow tissue 86 to easily acquire a sectioning position, i.e. move toward, to the right looking into the page, and elevated relative to Vibratome 82. The metal plate 90 is part of motorized stage 90, which is controlled electronically, as previously discussed. It is appreciated that reference to a left or a right (translational or X, Y) direction, as used herein, is in no event limiting and can be different in alternative implementations. For example, the Vibratome 82 may be located to the right of the tissue 86 in which case the tissue is moved to the right toward the Vibratome. The same applies to the vertical direction in that the tissue 86 lowered relative to Vibratome 82.

The optical path 108 is shown to include microscope 92, tube lens 98, galvo scanner 100, aperture 122, beam expander 102, dichroic 104 and lasers 106. Lasers 106 are a combination of two lasers each with serving as a distinct excitation source. The optical path 110 is shown to include the microscope 94, the dichroic 112, tube lenses 114, 116, and CMOS cameras 118 and 120.

In operation, two laser beams 106 are generated by the objective 108, each with a distinct wavelength. The two lasers 106 in FIG. 8 are shown to have 488 nano meter (nm) and 561 nano meter lasers although lasers with other wavelengths may be employed. The mirror 124 reflects the laser beam (of 561 nm, by way of example) and the dichroic 104 combines the two lenses at a 45-degree angle. The combined laser beam is than expanded by the beam expander 102 and the expanded laser beam travels through the aperture 122 to galvo scanner 100, which is used to generate the light sheet. The light sheet travels through the lenses 98 and 96 to the illuminating objective 108 to the tissue 86, contained in the block 88. The detection objective 110 is then used to detect fluorescent images.

After travelling through the lens 96, the laser beam arrives at the microscope 92, which in an exemplary embodiment and without limitation has a magnification of 10×. The microscope 92 delivers a line sheet to the tissue 86 and the microscope 94 is used to detect fluorescent images. The microscope 94, exemplary embodiment of the invention, has a magnification of 16x although other magnification powers may be employed. The laser beam from the microscope 94 is put through the dichroic 112 splitting the beam into two beams with each traveling through a respective emission filter, in the embodiment of FIG. 8 emission filter green and emission filter red to a respective CMOS camera 118 and 120. The two cameras 118 and 120 ultimately generate image data forming the 3D image of the tissue 86, under the direction of a processor, such as shown in FIG. 7. The tissue 86 is shown mounted on X, Y, Z motorized stages and controlled by a processor as shown in FIG. 8a.

The laser light of the objective 108, traveling past the beam expander 102 serves an excitation light to the tissue 86. In an exemplary implementation, the block 88 is cut by the Vibratome prior to the cutting of the tissue 86.

FIG. 8a shows the microscope imaging system of FIG. 8 coupled to a processing circuit. As shown in FIG. 8a, the processing circuit 130 is coupled to the scanner 100, the Vibratome 82, the motorized X, Y, Z stage 90, and the cameras 118 and 120. It is understood that the cameras 118 and 120 are merely an example of a suitable camera type and that others, such as a charge couple device (CCD) camera can be employed instead.

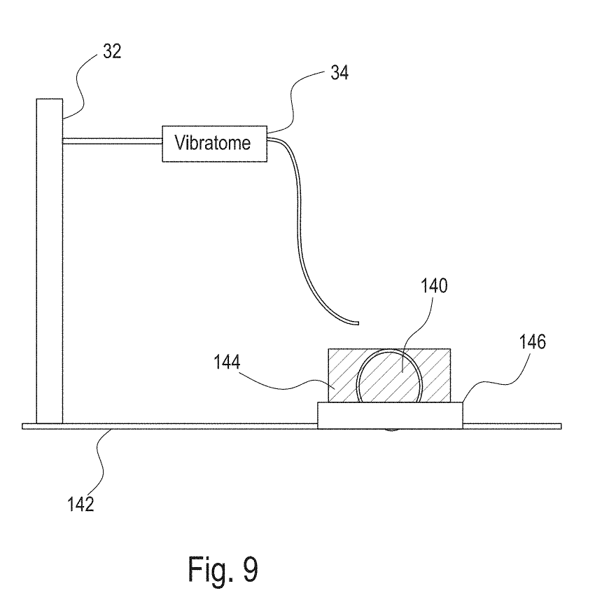

FIG. 9 shows a conceptual view of the Vibratome 34 mounted to the post 32 of FIG. 3. In FIG. 9, the tissue 140 is shown mounted to the stage 142 while housed in the block 144, which is positioned on the metal plate 146.

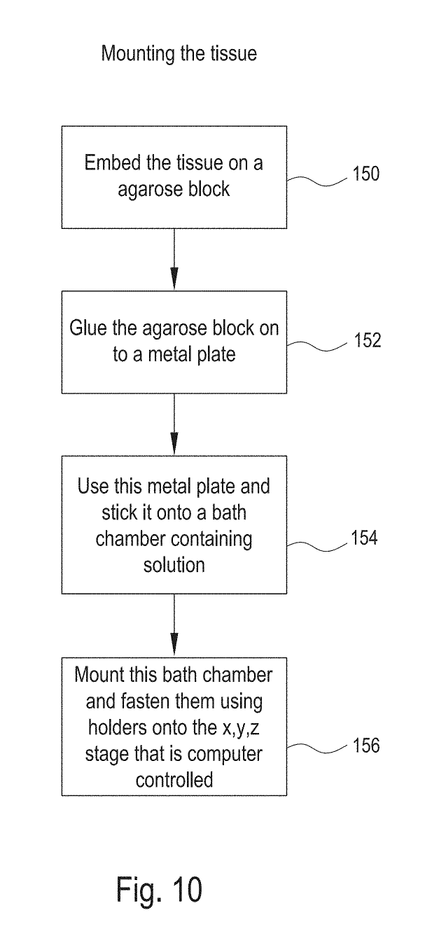

FIG. 10 shows a flow chart of some of the steps performed when mounting the tissue. At step 150, the tissue is embedded onto a block, an example of which is an agarose block. Next, at step 152, the Agarose block is glued onto the metal plate although techniques other than gluing may be employed for connecting the block to the metal plate. Agarose blocks are generally used to embed tissue, such as the tissue 86, while the cells of the tissue are processed for electron microscopic examination while the tissue is held in suspension.

Next, at step 154, using the metal plate, the tissue and the block are immersed in a bath chamber. The bath chamber contains a solution for “chemical clearing” solution. Next, at step 156, the bath chamber is mounted to the stage. The mounted bath chamber is then fastened to a X, Y, Z stage using holders with the stage being computer controlled.

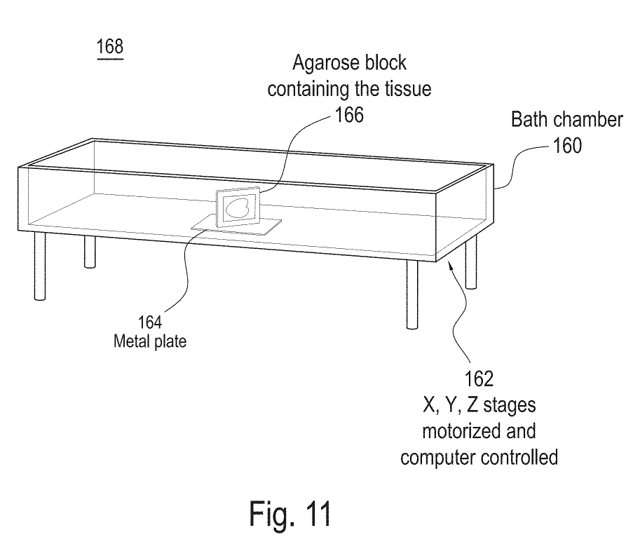

Consistent with the steps of FIG. 10, in FIG. 11, the tissue is shown mounted, in accordance with an exemplary implementation of the invention. In FIG. 11, the tissue 168 is shown contained in an agarose block 166, which is shown glued onto the metal plate 164. The metal plate 164, along with the block 166 and tissue 168 are mounted on the X, Y, Z motorized and computer controlled stages.

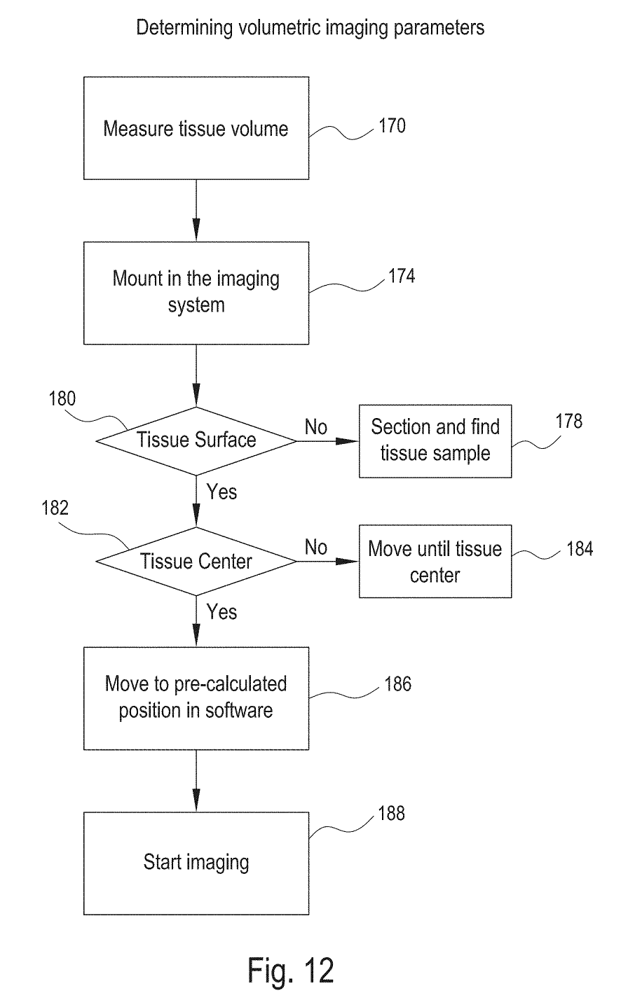

FIG. 12 shows a flow chart of some of the steps generally performed in determining volumetric imaging parameters at the outset of the imaging operation, such as step 108 of FIG. 1. The volumetric imaging parameters are optical parameters that are based on the optical path (mirrors, scanner, dichroic, lenses . . . ) of the imaging system in which they are employed. For instance, the optical path of the imaging systems of various exemplary implementations of the invention, some of which are shown and described herein, is made of optical pieces. A change in the optical pieces will result in different parameters of the light sheet. Commonly, different combination of lenses is employed to generate corresponding different parameters. In the various exemplary implementations, the optical parameters remain constant during operation.

Referring back to FIG. 12, at step 170, the volume of tissue is measured and next at step 174, the tissue is mounted in the imaging system, such as the imaging system 80 of FIGS. 8 and 8a. Next, at 180, it is determined whether or not the surface of the tissue facing the objectives has been found and if not, the process proceeds to step 178, otherwise, the process proceeds to 182. In the event the surface is not found, the tissue is sectioned to find the tissue surface, thus, the determination 180 and step 178 are repeated until the surface is found. At 182, a determination is made as to whether or not the center of the tissue is found and if not, the process proceeds to step 184 where the tissue is moved around until the center is found. Otherwise, the process proceeds to step 186. At step 186, the tissue is moved to a pre-calculated position, i.e. the position of the tissue as defined by the volumetric imaging parameters, under the control of software executed by the processor, such as processor 130 of FIG. 8a. After step 186, at step 188, the imaging system begins imaging.

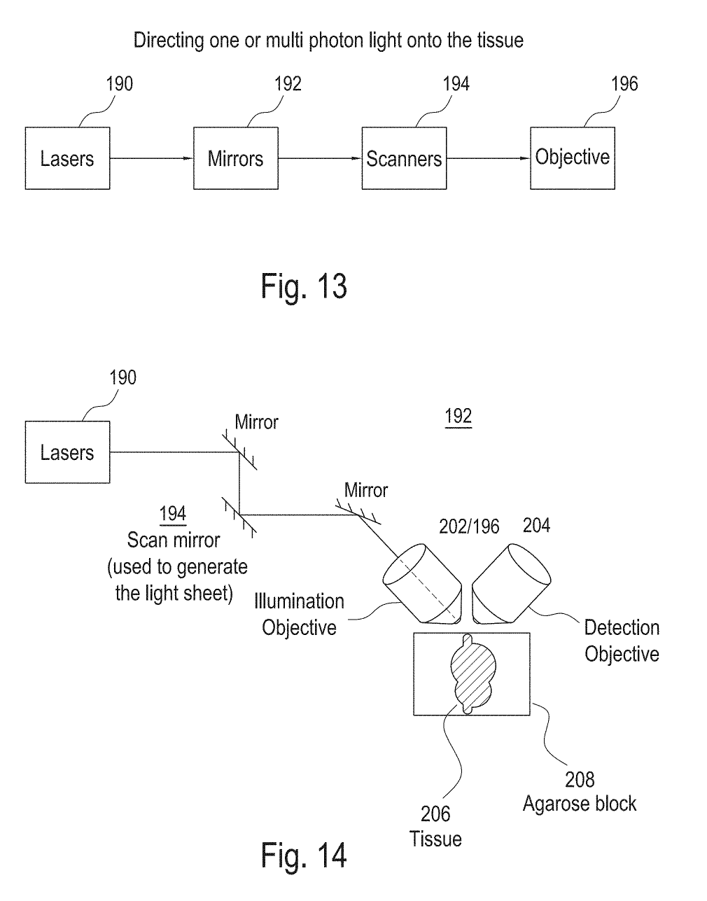

FIG. 13 shows a block diagram of some of the structures of the imaging system of an exemplary implementation. The imaging system 191 includes lasers 190, analogous to the lasers 106 of FIG. 8. Next, the laser beam travels through mirrors 192, such as the mirror 124 of FIG. 8, followed by scanners 194, such as the scanner 100 of FIG. 8, and finally the objective 196, such as the microscope 92 of the objective 108 of FIG. 8.

Analogous to the block diagram of FIG. 13, FIG. 14 shows, in conceptual form, a part of the imaging system of an exemplary implement of the invention. In FIG. 14, the lasers 190 generate laser beams that are combined into a single beam and employed to generate the fluorescent image through the mirrors 192 and scanner 194. The scanner 194 is used to generate the light sheet. The light sheet travels through another one of the mirrors 192 to the illuminating objective to the tissue 206 in the agarose block 208. The detection objective 204 is then used to detect fluorescent images.

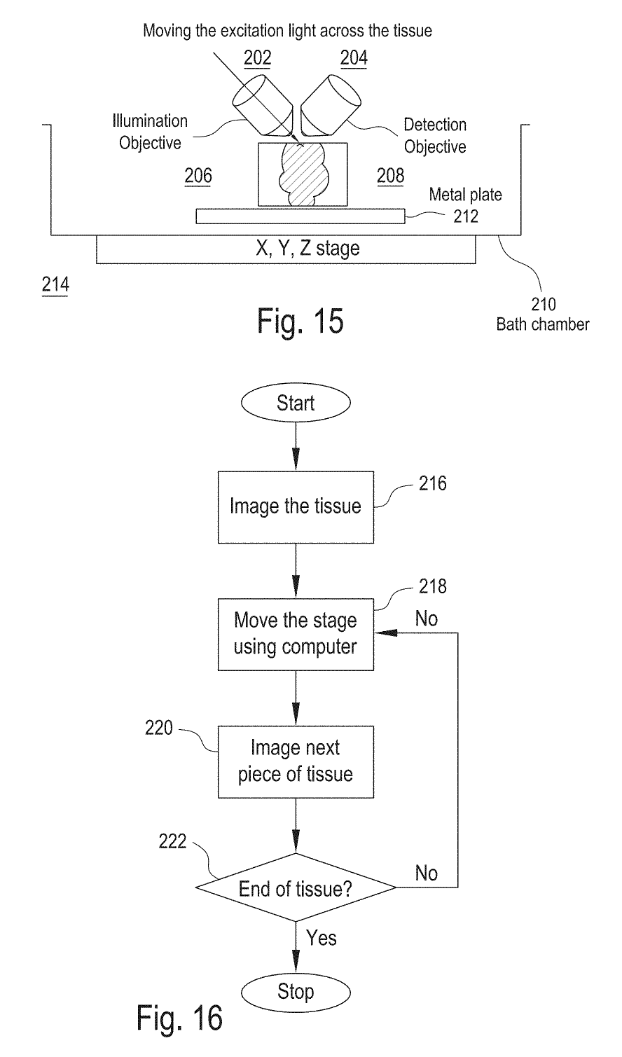

Similar to the relationship between FIGS. 13 and 14, FIGS. 15 and 16 show in conceptual form and in flow chart form, respectively, the process of moving the excitation light across the tissue during volumetric measurement. In FIG. 15, the illumination objective 202, the detection objective 204, the block 208, and tissue 206 are analogous to their counterparts in FIG. 14. Further shown in FIG. 15, the tissue 206 and block 208 are shown residing on the metal plate 212, which is attached to the X, Y, Z stage 214. The metal plate 212, block 208, and tissue 206 are shown immersed in the bath chamber 210.

Consistent with FIG. 15, in FIG. 16, at step 216, the tissue is imaged and at step 218, the tissue is moved as the result of the stage moving, under the control of the processor, toward the Vibratome. Next, at step 220, after the tissue has been sectioned, the next piece of tissue is imaged and at 222, the end of the tissue with all pieces having been imaged is reached and if not, the process repeats starting from step 218, otherwise, the process ends.

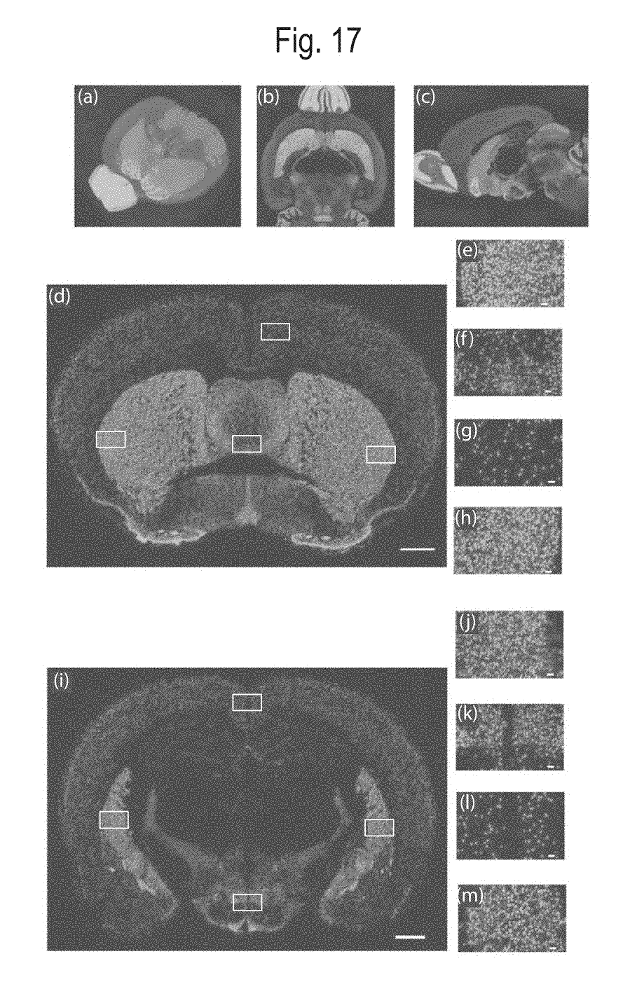

FIG. 17 shows various magnifications of 3D images of a mouse brain tissue generated by an imaging system of an implementations of the invention.

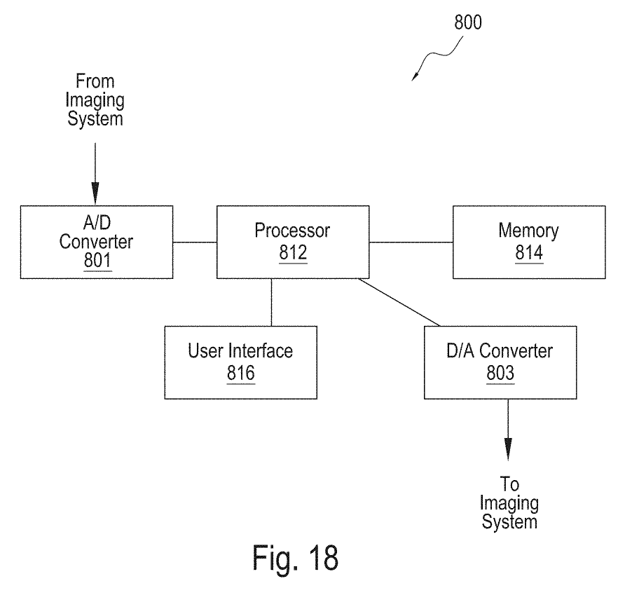

FIG. 18 shows a block diagram of the processor circuit of an implementation of the invention. The process circuit 800 is analogous to the processor circuit 130 of FIG. 8a. The processing circuit 800 is shown to include an analog-to-digital (A/D) converter 801, processor 812, user interface 816, digital-to-analog (D/A) converter 803, and memory 814.

In FIG. 18, the A/D converter 801 is shown to receive analog data, in the form of signals, such as imaging data, from an exemplary imaging system, such as the imaging system 30 of FIGS. 8 and 8a. The A/D converter 801 converts the analog signals to digital signals and couples the digital signals with the processor 812. Conversely, upon processing of signals, the digital-to-analog (D/A) converter 803 is employed converting the digital signals, from the processor 812, to analog signals and the analog signals are then transmitted to the imaging system 30.

In the example of FIG. 18, the computing device may enable functions of the microscope. In one example, the systems and methods can be implemented with a processor 812 and a memory 814, where the memory 814 stores instructions, which when executed by the processor 812, causes the processor 812 to perform the systems and methods. It can be appreciated that the components, devices or elements illustrated in and described may not be mandatory and thus some may be omitted in certain embodiments. Additionally, some embodiments may include further or different components, devices or elements beyond those illustrated.

In some example embodiments, the computing device may include processing circuitry 810 that is configurable to perform actions in accordance with one or more example embodiments disclosed herein. In this regard, the processing circuitry 810 may be configured to perform and/or control performance of one or more functionalities of the microscope. The processing circuitry 810 may be configured to perform data processing, application execution and/or other processing and management services according to one or more example embodiments. In some embodiments, the computing device or a portion(s) or component(s) thereof, such as the processing circuitry 810, may include one or more chipsets and/or other components that may be provided by integrated circuits.

In some example embodiments, the processing circuitry 810 may include a processor 812 and, in some embodiments, such as that illustrated, may further include memory 814. The processor 812 may be embodied in a variety of forms. For example, the processor 812 may be embodied as various hardware-based processing means such as a microprocessor, a coprocessor, a controller or various other computing or processing devices including integrated circuits such as, for example, an ASIC (application specific integrated circuit), an FPGA (field programmable gate array), some combination thereof, or the like. Although illustrated as a single processor, it can be appreciated that the processor 812 may include a plurality of processors. The plurality of processors may be in operative communication with each other and may be collectively configured to perform one or more functionalities of the computing device as described herein. In some example embodiments, the processor 812 may be configured to execute instructions that may be stored in the memory 814 or that may be otherwise accessible to the processor 812. As such, whether configured by hardware or by a combination of hardware and software, the processor 812 is capable of performing operations according to various embodiments while configured accordingly.

In some example embodiments, the memory 814 may include one or more memory devices. Memory 814 may include fixed and/or removable memory devices. In some embodiments, the memory 814 may provide a non-transitory computer-readable storage medium that may store computer program instructions that may be executed by the processor 812. In this regard, the memory 814 may be configured to store information, data, applications, instructions and/or the like for enabling the computing device to carry out various functions in accordance with one or more example embodiments. In some embodiments, the memory 814 may be in communication with one or more of the processor 812, the user interface 816 for passing information among components of the computing device.

FIGS. 19-21 show pictures of two tissues undergoing chemical clearing, in accordance with various implementations of the invention. In FIG. 19, the tissues 250 are at day 0 and as time progresses, as they undergo chemical cleaning, they become more translucent. At day 3, as shown in FIG. 20, the tissues look more translucent as shown by the state of tissues 250′. At day 8, the tissues are even more translucent, as shown by the state of the tissues 250″. Some examples of the chemical solution in which the tissues are immersed to undergo chemical cleaning are mCUBIC, Clarity, and ScaleA2, among a host of other suitable chemical solutions.

While various embodiments have been described, it can be apparent that many more embodiments and implementations are possible. Accordingly, the embodiments are not to be restricted.

The systems and methods described above may be implemented in many different ways in many different combinations of hardware, software firmware, or any combination thereof.

What is claimed is:

1. A microscope imaging system comprising:
a first optical path including a first objective with an associated first magnification;
a second optical path including a second objective with an associated second magnification, the first and second magnifications being distinct,
wherein the first and second microscopes are positioned to focus on a tissue to be imaged, the tissue having an associated tissue type and positioned on a motorized and moveable stage of the microscope imaging system,
further wherein the first objective serves as tissue-penetrating light-sheet and is configured to direct one or multi photon excitation lights onto the tissue from a position on top and at an oblique angle relative to the tissue,
further wherein the second objective is configured to collect fluorescent signals from the tissue upon detection of the tissue-penetrating light-sheet,
further wherein the one or multi photon excitation lights are directed across successive portions of the tissue with the number of successive portions being based, at least in part, on the tissue type;
an integrated Vibratome positioned in close proximately to the tissue and configured to:
section each successive portion of the tissue,
for each successive portion of the tissue, image across the sectioned successive portion of the tissue,
wherein upon imaging across each successive sectioned portion of the tissue, the one or multi photon excitation lights are moved across a next successive portion of the tissue;
a first image sensor, in the second optical path, configured to acquire a first image upon collection of the fluorescent image, the first image being defined by a first image data; and
a second image sensor, in the second optical path, configured to acquire a second image being defined by a second image data,
wherein the integrated Vibratome and the two optic paths cause better quality images of tissues with large volumes.

2. The microscope imaging system of claim 1, further including a processor circuit responsive to the first and second image data and configured to stitch the same to create a 3-Dimensional (3-D) image of the tissue.

3. The microscope imaging system of claim 2, wherein the motorized and moveable stage is moved under the direction of the processing circuit.

4. The microscope imaging system of claim 2, wherein the Vibratome sections the tissue under the direction of the processing circuit.

5. The microscope imaging system of claim 1, wherein the first and second image sensors are each a camera.

6. The microscope imaging system of claim 5, wherein the cameras are each of a CMOS or charge couple device (CCD) type.

7. The microscope imaging system of claim 6, wherein the cameras operate under the direction of the processor circuit.

8. The microscope imaging system of claim 1, wherein the tissue is substantially transparent by use of chemical clearance.

9. The microscope imaging system of claim 8, further including a bath chamber wherein imaging solution is used to cause the chemical clearance of the tissue.

10. The microscope imaging system of claim 1, wherein the one or multi photon excitation lights are generated from at least two lasers with distinct wavelengths.

11. The microscope imaging system of claim 1, wherein the tissue is positioned within a block, the block is positioned on top of a plate and the plate is attached to the motorized and moveable stage.

12. The microscope imaging system of claim 1, wherein the first objective includes a single-photon light-sheet microscope.

13. The microscope imaging system of claim 1, wherein the excitation light has a penetration depth in the tissue in the range of hundred micrometers or more because of a “chemical clearing” of the tissue by matching the refractive index of the tissue and the imaging solution.

14. The microscope imaging system of claim 1, wherein the excitation light has a penetration depth in the tissue in the range of hundred micrometers or more because of the use of multi-photon microscopy excitation.

15. The microscope imaging system of claim 1, wherein the first objective comprises a multi-photon light-sheet microscope.

16. A method of imaging tissue comprising:
using an illumination objective, directing one or multi photon excitation lights onto a portion of a tissue from a position on top and at an oblique angle relative to the tissue, the tissue mounted on a stage and made of more than one portion;
generating a tissue-penetrating light-sheet from the one or multi photon excitation lights;
using a detection objective, detecting the tissue-penetrating light-sheet;
upon detecting the tissue-penetrating light-sheet, using the detection objective, collecting fluorescent signals from the tissue;
acquiring a first image of the tissue, in an imaging position, the first image defined by a first image data;
acquiring a second image of the tissue, in the imaging position, the second image defined by a second image data;
moving the tissue to a sectioning position;
using an integrated Vibratome, sectioning the portion of the tissue; and
stitching the first and second images to for a 3-D image of the tissue; and
repeating the acquiring steps through the stitching step and until all portions of the tissue have been sectioned and imaged.

17. The method of imaging tissue of claim 16, further including chemical clearing the tissue prior to starting the sectioning.

18. The method of imaging tissue of claim 16, further including moving the stage to find a center of a top surface of the tissue, prior to sectioning.

19. The method of imaging tissue of claim 16, further including acquiring images with a charge coupled device or CMOS imaging device.

20. The method of imaging tissue of claim 16, further including determining volumetric imaging parameters prior to starting the sectioning.

21. The method of imaging tissue of claim 20, wherein the volumetric imaging parameters are kept constant throughout the steps of claim 1.

22. The method of imaging tissue of claim 16, further including controlling moving the stage and Vibratome using a processor.
